(19) United States
(12) Patent Application Publication
(10) Pub. No.: US 2012/0283756 A1
(43) Pub. Date: Nov. 8, 2012

(54) TREATMENT OF HYPERTENSION AND HEART DISEASE VIA SURGERY OF THE STOMACH

(75) Inventors: Scott Moonly, Irvine, CA (US); John Cox, San Clemente, CA (US); Richard C. Ewers, Fullerton, CA (US)

(73) Assignee: USGI MEDICAL, INC., San Clemente, CA (US)

(21) Appl. No.: 13/479,991

(22) Filed: May 24, 2012

Related U.S. Application Data
(63) Continuation-in-part of application No. 12/409,335, filed on Mar. 23, 2009.
Application No. 12/409,335 is a continuation-in-part of application No. 11/070,863, filed on Mar. 1, 2005, now Pat. No. 8,216,252.
Application No. 11/070,863 is a continuation-in-part of application No. 10/840,950, filed on May 7, 2004.
Application No. 10/840,950 is a continuation-in-part of application No. 10/735,030, filed on Dec. 12, 2003.
Application No. 10/735,030 is a continuation-in-part of application No. 10/639,162, filed on Aug. 11, 2003, now Pat. No. 7,618,426.
(60) Provisional application No. 61/038,487, filed on Mar. 21, 2008.

(51) Int. Cl. A61B 17/00; A61B 17/03

(52) U.S. Cl. 606/151; 128/898

(57) ABSTRACT

Devices and methods for endolumenally manipulating stomach fundus tissue alter the function of nearby nerves. The altered function of the nerves interacts with the cardiopulmonary system to cause a substantially permanent reduction in blood pressure. The altered nerve function may also treat heart disease as well. This application also relates to devices and methods for endolumenally manipulating stomach tissue to alter hormone production from cells associated with stomach tissue, providing a therapeutic effect in treating hypertension and heart disease, not conventionally associated with the stomach.

### REFERENCE TO RELATED APPLICATIONS

This Application is a Continuation-in-Part of U.S. patent application Ser. No. 12/409,335 filed on Mar. 23, 2009, and now pending, which claims priority to U.S. patent application Ser. No. 61/038,487 filed on Mar. 21, 2008.

This Application is also a Continuation-in-Part of U.S. patent application Ser. No. 11/070,863 filed on Mar. 1, 2005 and now pending, which claims priority to U.S. patent application Ser. No. 10/840,950 filed on May 7, 2004 and now pending.

This Application is also a Continuation-in-Part of U.S. patent application Ser. No. 10/735,030 filed on Dec. 12, 2003 now pending, which is a Continuation-in-Part of U.S. patent application Ser. No. 10,639,162, filed Aug. 11, 2003, now U.S. Pat. No. 7,618,426. Each of the Applications listed above is incorporated herein by reference.

### BACKGROUND OF THE INVENTION

Hypertension or high blood pressure is a common chronic medical condition where the blood pressure on arteries is elevated beyond normal ranges. In the United States, almost 25% of the adult population has hypertension. Hypertension generally has no symptoms. Hypertensive patients consequently may go indefinitely without knowing of their condition. While symptom-free, hypertension is a serious condition because it is a primary risk factor for stroke, heart attack, aneurysms, and peripheral arterial disease, among others. Even at lower levels of severity, hypertension tends to shorten life expectancy.

Hypertension can sometimes be treated using only changes in lifestyle, such as changes in diet, weight loss and physical exercise. However, these steps alone often are not sufficient. Various drugs can then be used for hypertension treatment, typically indefinitely, and on a daily basis. Unfortunately, in some patients, drugs have limited effect. Improved techniques are therefore needed for treating hypertension.

Heart disease, heart failure, and disorders of the circulatory system are often related to hypertension. These diseases or conditions may include or be associated with atherosclerosis, cardiac arrhythmias, congestive heart failure, and others. Treating these conditions is also a challenge for medical science.

Although the stomach may be conventionally thought off as unrelated to the circulatory system and the heart, there is substantial evidence showing direct interaction between them. the stomach is known to be involved in the regulation of other physiologic processes. For example, bariatric surgery has a significant impact on long-term cardiovascular events. Specifically, it has been found that bariatric surgery is associated with a not only a reduced number of cardiovascular deaths, but also reductions in strokes and myocardial infarctions.

It is also known from scientific literature that in addition to its functions in the digestive process, the human stomach also acts as an initiator or catalyst for a wide variety of chemical and hormonal changes before, during and after a meal. The stomach is surrounded by parasympathetic (stimulant) and orthosympathetic (inhibitor) plexuses. These are networks of blood vessels and nerves in the anterior gastric, posterior, superior and inferior, celiac and myenteric, which regulate both the secretions activity and the motion activity of stomach muscles. The movement and the flow of chemicals into the stomach are controlled by both the autonomic nervous system and by the various digestive system hormones. With recognition of the interactions between the stomach and other body systems and organs, the inventive methods described below provide treatments for hypertension and heart disease via surgery of the stomach.

### SUMMARY OF THE INVENTION

Recent studies suggest that there is a direct relationship between the gastrointestinal and cardiovascular systems, with gastrointestinal function thought to have a direct influence on blood pressure. Some studies discuss gastric distension, or tensioning of stomach tissue, as causing an increase in blood pressure, while others find a decrease in blood pressure. It is also known that activation of the vagus nerve endings in the stomach typically leads to a reduction in heart rate, blood pressure, or both. The effectiveness, duration, underlying causes, and other factors concerning changes in blood pressure relative to a condition in the stomach appear to be the subject to ongoing research and are currently not fully understood.

At the same time, recently developed surgical techniques now enable the equivalent of essentially permanent gastric distension, specifically via endolumenal stomach surgery. The inventors of these endoluminal surgical techniques of the stomach have now in turn discovered that, surprisingly, hypertension and other diseases unrelated to the stomach may be treated via surgery of the stomach. Specifically, the inventors have discovered that hypertension may be treated by placing plications in the fundus of the stomach, leading to a substantially permanent reduction in blood pressure, and that use of plications can similarly be useful in treating heart disease.

This application is directed to devices and methods for endolumenally manipulating stomach tissue to alter the function of nerves located in or near stomach tissue. The altered function of the nerves interacts with the cardiopulmonary system to cause a substantially permanent reduction in blood pressure. The altered nerve function may also treat heart disease. This application also relates to devices and methods for endolumenally manipulating stomach tissue to alter hormone production from cells associated with stomach tissue, providing a therapeutic effect in treating conditions and diseases not conventionally associated with the stomach.

In one method, a delivery catheter is advanced through a patient's mouth and esophagus and into the patient's stomach. The delivery catheter includes a flexible tube having a needle at its distal end and with a first tissue anchor assembly contained within the flexible tube. A grasping/pulling instrument is used to form a first tissue fold in the tissue of the stomach fundus. The tissue fold may have a serosa-to-serosa contact of tissue on the peritoneal surface of the stomach fundus. A needle is passed through the tissue fold, typically while the grasper is holding the tissue the fold. A first tissue anchor assembly attached to suture is deployed from the delivery catheter on a first side of the tissue fold. The needle is then withdrawn from the tissue fold. A second tissue anchor assembly slidable along the suture is then deployed from the delivery catheter on the second or opposite side of the tissue fold.

The second tissue anchor assembly and a one-way cinch device are pushed up against the second side of the tissue fold. The cinch is designed to resist reverse movement along the suture. Accordingly, the cinch holds the second anchor assembly against the second side of the tissue fold. The suture passing through the tissue fold holds the anchor assemblies securely against the sides of the tissue fold. The suture leading back through the delivery catheter is then cut near the cinch, leaving the first and second tissue anchors in place to substantially permanently maintain the tissue fold. Additional tissue folds may be made by repeating these steps.

The tissue folds in the fundus change the way nerves located in or near stomach tissue interact with the cardiopulmonary system to cause a substantially permanent reduction in blood pressure.

Other objects, features and advantages will become apparent from the following detailed description. The invention resides as well in sub combinations of the method steps and apparatus elements described below.

### BRIEF DESCRIPTION OF THE DRAWINGS

FIG. 1 is an illustration of an endolumenal system advanced endolumenally into a stomach.

FIG. 2 is an exploded view of the tissue manipulation assembly and the tissue anchor assembly delivery device shown in the system of FIG. 1.

FIG. 3 is an exploded view of a tissue anchor assembly delivery device shown in FIG. 2.

FIGS. 4A through 4C are enlarged side views of the tissue manipulation assembly and helical tissue engagement instrument of the system shown in FIG. 1.

FIG. 5 is a schematic representation of a tissue anchor assembly included in the tissue anchor assembly delivery device shown in FIG. 3.

FIG. 6 is a schematic representation of the tissue anchor assembly of FIG. 5 securing a tissue fold.

FIGS. 7A-7F are illustrations showing a progression of an endolumenal procedure for treating hypertension.

### DETAILED DESCRIPTION

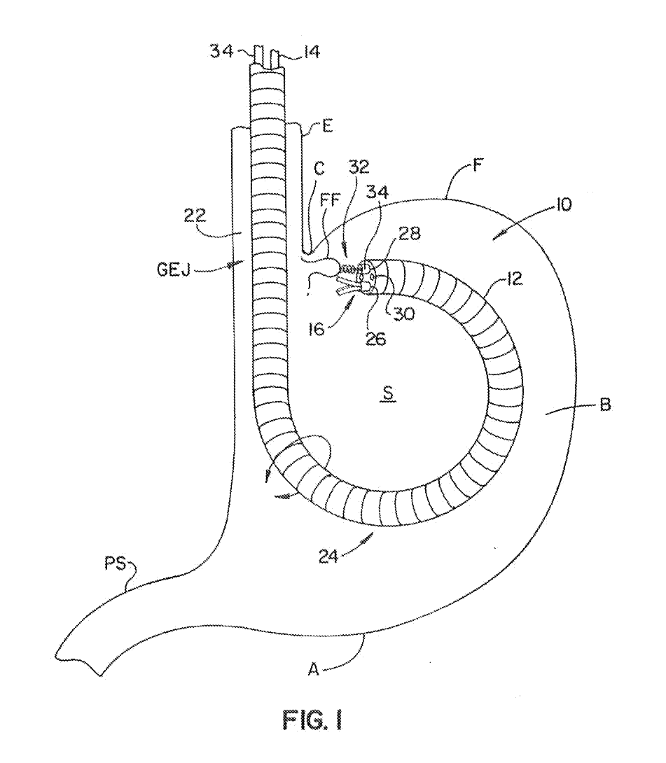

The anatomy of the stomach can be divided into different segments on the basis of the mucosal cell types in relation to external anatomical boundaries. As shown in FIG. 1, the cardiac segment C is immediately subjacent to the gastroesophageal junction GEJ and is a transition zone of the esophageal squamous epithelium into the gastric mucosa. The fundus F is the portion of the stomach that extends above the gastroesophageal junction. The body B or corpus of the stomach extends from the fundus F to the incisura angularis on the lesser curvature of the stomach. The majority of parietal acid forming cells are present in this segment. The fundus F and the body B function as the main reservoir of ingested food. The antrum A extends from the lower border of the body B to the pyloric sphincter PS. The majority of gastrin producing or G-cells are present in the antral mucosa.

For the most part, gastric innervation is provided by the vagus nerves which form a plexus around the esophagus and then reform into vagal trunks above the esophageal haitus. An extensive myenteric plexus is formed within the muscular wall of the stomach. Impulses from stretch or tension receptors within the gastric wall are transmitted to the nucleus tractus solitaris of the brain stem by afferent vagal fibers. These stretch/tension receptors within the fundus F and body B detect gastric distension or gastric pressure from ingested food. In the methods described here, a treatment for hypertension or heart disease may be provided by modulating mechanical or chemical receptors in the stomach or small bowel that impact the function of the cardiovascular system. These receptors include pressure receptors, sodium receptors; stretch receptors and other mechanical receptors; chemical receptors and RAS associated receptors (i.e. AT1 and AT2). Modulation of receptors may be achieved by forming plications in the stomach or small bowel.

The gastrointestinal lumen, including the stomach, includes four tissue layers. The mucosa layer is the top tissue layer followed by connective tissue, the muscularis layer and the serosa layer. When plicating from the peritoneal side of the GI tract, it is easier to gain access to the serosal layer. In endolumenal approaches to surgery, the mucosa layer is visible via an endoscope. The muscularis and serosal layers are difficult to access because they are only loosely adhered to the mucosal layer. To create a durable tissue fold or plication with suture and anchors, it is preferable to have serosa to serosa contact in the tissue fold. The mucosa and connective tissue layers typically do not heal together in a way that can sustain the tensile loads imposed by normal movement of the stomach wall during ingestion and processing of food. Folding the serosal layers with serosa-to-serosa contact allows the tissue to heal together and form a durable tissue fold, plication, or elongated invagination.

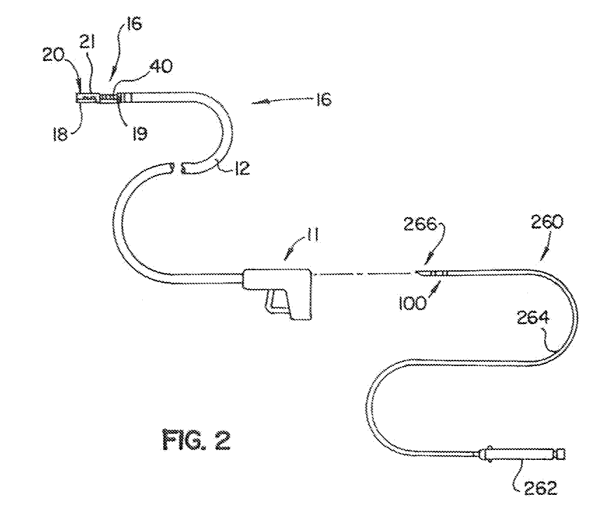

Turning now to the drawings, in FIG. 1 and FIG. 2, and endoluminal system 10 includes an endoscopic body 12 having a covering 22 and a steerable distal portion 24. The endoscopic body 12 may have at least first and second lumens 26, 28, respectively. Additional lumens may be provided through the endoscopic body 12, such as a visualization lumen 30, through which an endoscope may be positioned to provide visualization. Alternatively, an imager such as a CCD imager or optical fibers may be provided in lumen 30 to provide visualization. An optional thin wall sheath may be disposed through the patient's mouth, esophagus E, and possibly past the gastroesophageal junction GEJ into the stomach S.

Referring still to FIGS. 1 and 2, the endoluminal system includes a tissue manipulation assembly 16 and a tissue anchor deployment assembly 260. The tissue manipulation assembly 16 includes a flexible catheter or tubular body 12 which is sufficiently flexible for advancement into a body lumen, e.g., transorally, percutaneously, laparoscopically, etc. The tubular body 12 is torqueable through various methods, e.g., utilizing a braided tubular construction, such that when a handle 11 is manipulated and/or rotated by a practitioner from outside the patient's body, the longitudinal and/or torquing force is transmitted along the body 12 such that the distal end of the body 12 is advanced, withdrawn, or rotated in a corresponding manner. Jaws 18 and 20 are attached to the front end of the body 12, optionally at a pivot joint connection 19.

A launch tube 40 extends through the body 12 may be pivotally attached to the upper jaw. The front end of the launch tube may be designed to change from straight into a curved or arcuate shape when the launch tube is advanced forward. When in the curved shape, the launch tube opening may be generally perpendicular to the upper jaw 20. The launch tube 40, or at least the exposed portion of the launch tube 40, may be fabricated from a highly flexible material or it may be fabricated, e.g., from Nitinol tubing material which is adapted to flex, e.g., via circumferential slots, to permit bending. Movement of the launch tube may also open and close the jaws. Using the launch tube 40 to articulate the jaws eliminates the need for a separate jaw moving mechanism.

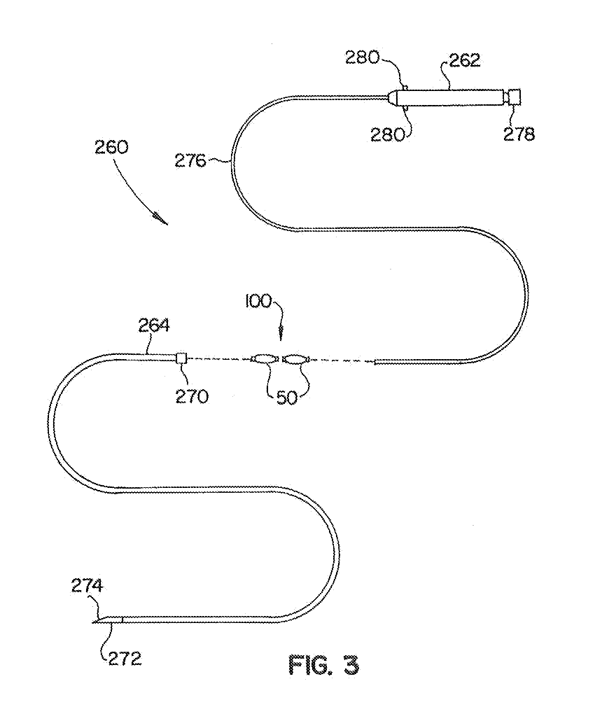

As shown in FIG. 3, the tissue anchor assembly delivery system 260 may be deployed through the tissue manipulation assembly 16 by sliding it in through the handle 11 and through the tubular body 12. Once the needle 272 has been advanced through the tissue fold FF, the first anchor assembly 100 may be deployed or ejected. The anchor assembly 100 is normally positioned within the distal portion of a tubular sheath 264. Once the anchor assembly 100 has been fully deployed from the sheath 264, the spent tissue anchor assembly delivery system 260 may be removed and replaced from the tissue manipulation assembly 16 without having to remove the tissue manipulation assembly 16 from the patient.

The sheath or catheter 264 and the housing 262 may be interconnected via an interlock 270 which may be adapted to allow for the securement as well as the rapid release of the sheath 264 from the housing 262 through any number of fastening methods, e.g., threaded connection, press-fit, releasable pin, etc.

A pusher 276 which may be a flexible wire or tube within the sheath slides within the housing 262. An actuator 278 on the housing 262 is used to slide the pusher 276 relative to the sheath 264, to push anchors out from the opening 274 at the tip of the needle 272. Needle assembly guides 280 may protrude from the housing 262 for guidance through the locking mechanism.

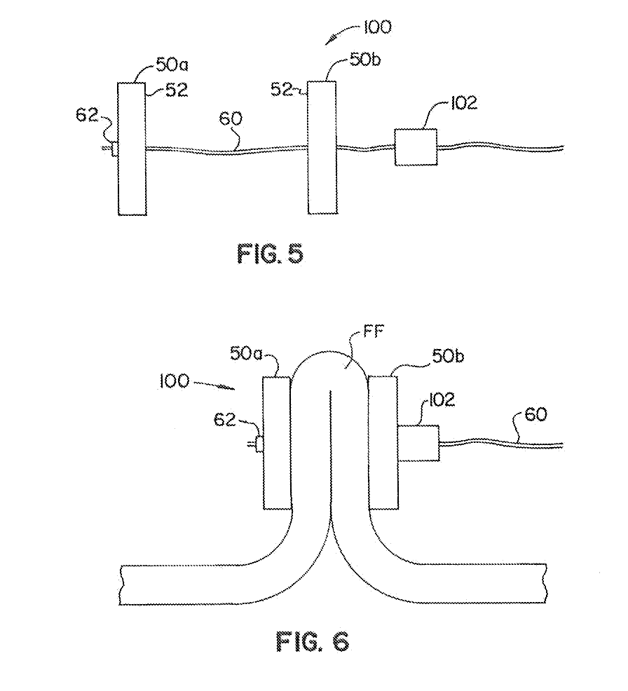

As shown in FIG. 5, typically, the tissue anchor assemblies include a pair of tissue anchors 50a and 50b, slidably attached to a suture 60. A knot 62 or other protrusion on the distal end of the suture keeps the distal anchor assembly from sliding off the end of the suture 60. The suture runs back up through the catheter 264 to the control handle 262, so that after both anchor assemblies have been deployed, the surgeon can tension the suture. A locking mechanism, such as a cinch 102, is also slidably retained on the suture 60. The cinch 102 is configured to provide a cinching force against the anchors to impart a tension force on the suture. With the suture under tension, the proximal anchor assembly 50b and the cinch 102 are pushed up against the fold FF. Accordingly, the tissue anchor assembly 100 is adapted to hold a fold of tissue, as shown in FIG. 6.

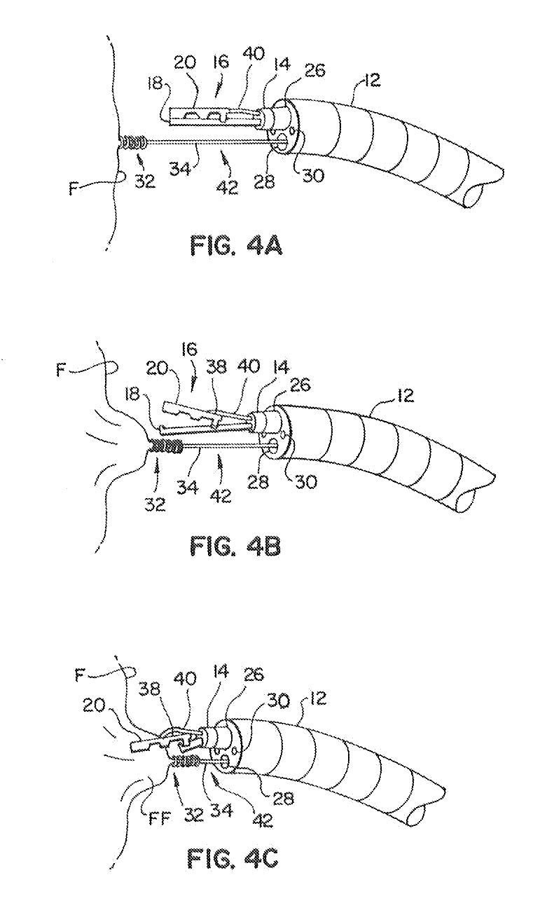

Surgery on the fundus to treat hypertension or heart disease may be performed as follows. The fundus F may be visualized through the visualization lumen 30 or a separate imager. In either case, the tissue manipulation assembly 16 and the tissue engagement member 32 may be advanced distally out from the endoscopic body 12 through lumens 26, 28. The distal steerable portion 24 of the endoscopic body 12 is steered to an orientation to position the jaws to engage the fundus. FIG. 1 shows a tissue manipulation assembly 16 advanced through the first lumen 26 and a helical tissue engagement member 32 positioned upon a flexible shaft 34 advanced through the second lumen 28. To obtain a durable tissue fold FF, the engagement member 32 is advanced or corkscrewed into fundus tissue. The jaws are opened, optionally by pulling launch tube 40 back as shown in FIG. 4B.

The engagement member 32 is then pulled back to draw the engaged tissue FF between the jaws 18 and 20, as shown in FIG. 4C. Once the tissue has been pulled or manipulated between the jaws, the jaws are closed, in this case by pushing the launch tube 40 forward. Movement of the launch tube may also change the angle of the jaws and the front end of the launch tube relative to the tissue.

With the tissue engaged between the jaws 18, 20, a needle assembly may be fed through the handle with the needle 272 moving out of the front end of the launch tube 40. The needle 272 pierces through the engaged tissue fold FF. The pusher is then used to push out the first anchor. The needle 272 is then pulled back through the tissue fold FF and the second anchor is deployed. The cinch and the second anchor are pushed up against the tissue fold FF, using the jaws or another instrument, to form a permanent tissue fold.

Using the methods described above, permanent tissue folds or plications FF may be made in the fundus to treat hypertension and heart disease. Plications. made in or on the fundus near the location of the vagal nerve branch (anterior, major) have the effect of compressing the wall and changing the effectiveness of the nerve branch, thereby inducing lowering blood pressure.

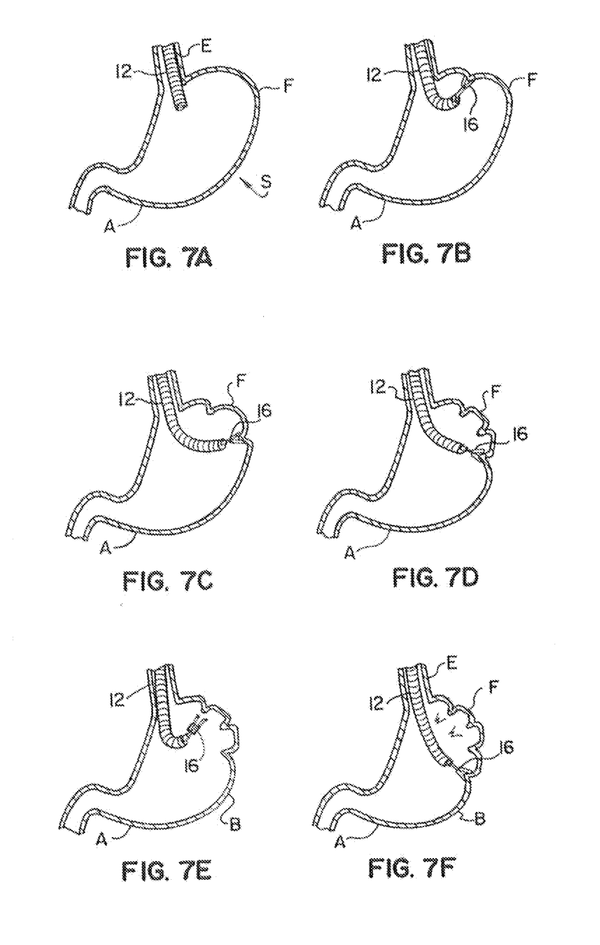

Turning to FIGS. 7A-7F, additional tissue folds FF may optionally be made. These additional folds FF may be substantially aligned in a first row extending through a portion of the fundus F, as shown in FIGS. 7C and 7D. These may be in a random pattern or in rows or other patterns in the fundus F, as shown in FIGS. 7E and 7F, thereby acting on nerves at the stomach, which in turn can act to treat diseases thought of as unrelated to the stomach, such as hypertension and heart disease. The mid-body B posterior inner wall of the stomach. and the antral region A may be left substantially unaltered.

Although the methods above are described as endoluminal trans-oral methods, these same methods may be performed in other ways as well, such as trans-anally, percutaneously, laporoscopically, robotically, or even via traditional open body surgery.

Thus, novel systems and methods have been shown and described. Various changes and substitutions may of course be made without departing from the spirit and scope of the invention. The invention, therefore, should not be limited, except by the following claims and their equivalents.

What is claimed is:

1. A method for treating hypertension or heart disease in a human patient, comprising:
forming one or more substantially permanent plications in the tissue of the stomach; and
the plications altering the function of nerves located in or near the stomach and/or altering hormone production from cells associated with stomach tissue, with the altering providing a therapeutic disease treating effect on the patient.

2. A method for treating hypertension or heart disease in a human patient, comprising:
forming one or more substantially permanent plications in the tissue of the stomach; and
the plications altering the function of nerves located in or near the stomach, with the altering providing a therapeutic disease treating effect on the patient.

3. A method for treating hypertension or heart disease in a human patient, comprising:
forming one or more substantially permanent plications in the tissue of the stomach; and
the plications altering hormone production from cells associated with stomach tissue, with the altering providing a therapeutic hypertension or heart disease treating effect on the patient.

4. A method for treating hypertension in a human patient, comprising:
forming one or more substantially permanent plications in the tissue of the stomach; and
the plications altering the function of nerves located adjacent to the fundus, with the altering providing a substantially permanent reduction in blood pressure in the patient.

5. A method for treating hypertension, comprising:
a) advancing a delivery catheter through a patient's mouth and esophagus and into the patient's stomach;
b) forming a tissue fold in the tissue of the stomach fundus;
c) passing the needle through the tissue fold;
d) deploying a first tissue anchor assembly from the needle on a distal side of the tissue fold;
e) withdrawing the needle back through the tissue fold;
deploying a second tissue anchor assembly from the needle on a proximal side of the tissue fold, with the second tissue anchor assembly linked to the first tissue anchor assembly by a suture; and
g) securing the second tissue anchor assembly in place to form a substantially permanent plication in the fundus;
h) with the plication altering the function of nerves located in or near the stomach to cause a reduction in the patient's blood pressure.

6. The method of claim 5 further comprising forming additional plications in the fundus by repeating at least steps b-g.

7. The method of claim 5 with the tissue fold including a serosa-to-serosa contact of tissue on the peritoneal surface of the stomach fundus.

8. The method of claim 5 further comprising forming additional plications in the fundus by repeating at least steps b-g.

9. The method of claim 5 with the tissue fold including a serosa-to-serosa contact of tissue on the peritoneal surface of the stomach fundus.

10. The method of claim 1 with the plications formed in the fundus.

11. The method of claim 2 with the plications formed in the fundus.

12. The method of claim 3 with the plications formed in the fundus.
